(19) United States
(12) Patent Application Publication
(10) Pub. No.: US 2004/0120926 A1
(43) Pub. Date: Jun. 24, 2004

(54) Reactive oxygen metabolite inhibitors for use in compositions and methods of treatment

(76) Inventors: Kristoffer Hellstrand, Goteborg (SE); Svante Hermodsson, Molndal (SE); Kurt R. Gehlsen, Encinitas, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/680,865

(22) Filed: Oct. 7, 2003

Related U.S. Application Data
(63) Continuation-in-part of application No. 09/616,622, filed on Jul. 14, 2000.
(60) Provisional application No. 60/144,394, filed on Jul. 16, 1999.

(51) Int. Cl. A61K 38/21; A61K 39/00; A61K 31/7048; A61K 31/353; A61K 31/02

(52) U.S. Cl. 424/85.2; 424/85.5; 424/85.6; 424/85.7; 424/184.1; 514/27; 514/456; 514/743

(57) ABSTRACT

Compositions and methods for treating a variety of conditions in which a reactive oxygen metabolite (ROM) inhibitor or scavenger is administered alone or in conjunction with additional agents. Such conditions include, cancer, viral diseases, and inflammatory diseases, for example.

### RELATED APPLICATIONS

This application is a continuation-in-part of U.S. application Ser. No. 09/616,622, filed Jul. 14, 2000, which claims priority to U.S. Provisional Patent Application No. 60/144,394, filed on Jul. 16, 1999, both of which are expressly incorporated by reference in their entireties.

### BACKGROUND OF THE INVENTION

1. Field of the Invention

The invention disclosed herein relates to compositions and methods of treating a variety of conditions in which a reactive oxygen metabolite (ROM) inhibitor or scavenger is administered alone or in conjunction with additional agents. Such conditions include, cancer, viral diseases, and inflammatory diseases, for example. The administration of these various agents results in the activation and protection of cytotoxic lymphocytes from the deleterious and inhibitory effects of monocytes/macrophages (MO), as well as a stimulation of the anti-cancer and anti-viral properties of cytotoxic lymphocytes. In addition, antigen presenting cells may become more effective at antigen presentation to certain cytotoxic lymphocytes as a direct effect of ROM inhibitor administration. The addition of other agents that are cytotoxic lymphocyte activation compounds that stimulate the cytotoxic activity of these lymphocytes, preferably in a synergistic fashion with a ROM inhibitor, are also contemplated. Representatives of such immunological stimulatory compounds include cytokines, peptides, flavonoids, vaccines, and vaccine adjuvants. Additional classes of agents usable with the methods herein encompass chemotherapeutic and/or antiviral agents. The teachings herein also contemplate the use of reactive oxygen metabolite scavengers in conjunction with the above-mentioned compounds.

2. Description of the Related Art

The immune system has evolved complex mechanisms for recognizing and destroying foreign cells or organisms present in the body of the host. Harnessing the body's immune mechanisms is an attractive approach to achieving effective treatment of malignancies and viral infections.

The immune system has two types of responses to foreign bodies based on the components which mediate the response: a humoral response and a cell-mediated response. The humoral response is mediated by antibodies while the cell-mediated response involves cells classified as lymphocytes. Recent anticancer and antiviral strategies have focused on utilizing the cell-mediated host immune system as a means of anticancer or antiviral treatment or therapy. A brief review of the immune system will assist in placing the teachings herein in context.

Generation of an Immune Response

The immune system functions in three phases to protect the host from foreign bodies: the cognitive phase, the activation phase, and the effector phase. In the cognitive phase, the immune system recognizes and signals the presence of a foreign antigen or invader in the body. The foreign antigen can be, for example, a cell surface marker from a neoplastic cell or a viral protein. Once the system is aware of an invading body, the cells of the immune system proliferate and differentiate in response to the invader-triggered signals. The last stage is the effector stage in which the effector cells of the immune system respond to and neutralize the detected invader.

A wide array of effector cells implement an immune response to an invader. One type of effector cell, the B cell, generates antibodies targeted against foreign antigens encountered by the host. In combination with the complement system, antibodies direct the destruction of cells or organisms bearing the targeted antigen.

Another type of effector cell is the cytotoxic lymphocyte. The natural killer cell (NK cell), a type of cytotoxic lymphocyte having the capacity to spontaneously recognize and destroy a variety of virus infected cells as well as malignant cell types. The method used by NK cells to recognize target cells is poorly understood.

Another type of cytotoxic lymphocyte is the T-cell. T-cells are divided into three subcategories, each playing a different role in the immune response. Helper T-cells secrete cytokines which stimulate the proliferation of other cells necessary for mounting an effective immune response, while suppressor T-cells down regulate the immune response. A third category of T-cell, the cytotoxic T-cell (CTL), is capable of directly lysing a targeted cell presenting a foreign antigen on its surface.

The Major Histocompatability Complex and T Cell Target Recognition

T-cells are antigen specific immune cells that function in response to specific antigen signals. B lymphocytes and the antibodies they produce are also antigen specific entities. However, unlike B lymphocytes, T-cells do not respond to antigens in a free or soluble form. For a T-cell to respond to an antigen, it requires the antigen to be bound to a presenting complex known as the major histocompatibility complex (MHC).

MHC complex proteins provide the means by which T-cells differentiate native or “self” cells from foreign cells. There are two types of MHC, class I MHC and class II MHC. T Helper cells (CD4⁺) predominately interact with class II MHC proteins while cytolytic T-cells (CD8⁺) predominately interact with class I MHC proteins. Both MHC complexes are transmembrane proteins with a majority of their structure on the external surface of the cell. Additionally, both classes of MHC have a peptide binding cleft on their external portions. It is in this cleft that small fragments of proteins, native or foreign, are bound and presented to the extracellular environment.

Cells called antigen presenting cells (APCs) display antigens to T-cells using the MHC complexes. For T-cells to recognize an antigen, it must be presented on the MHC complex for recognition. This requirement is called MHC restriction and it is the mechanism by which T-cells differentiate “self” from “non-self” cells. If an antigen is not displayed by a recognizable MHC complex, the T-cell will not recognize and act on the antigen signal.

T-cells specific for the peptide bound to a recognizable MHC complex bind to these MHC-peptide complexes and proceed to the next stage of the immune response.

Cytokines Involved In Mediating the Immune Response

The interplay between the various effector cells listed above is influenced by the activities of a wide variety of chemical factors which serve to enhance or reduce the immune response as needed. Such chemical modulators may be produced by the effector cells themselves and may influence the activity of immune cells of the same or different type as the factor producing cell.

One category of chemical mediators of the immune response is cytokines, molecules which stimulate a proliferative response in the cellular components of the immune system.

Interleukin-2 (IL-2) is a cytokine synthesized by T-cells which was first identified in conjunction with its role in the expansion of T-cells in response to an antigen (Smith, K. A. Science 240:1169 (1988)). It is well known that IL-2 secretion is necessary for the full development of cytotoxic effector T-cells (CTLs), which play an important role in the host defense against viruses. Several studies have also demonstrated that IL-2 has antitumor effects that make it an attractive agent for treating malignancies (see e.g. Lotze, M. T. et al, in “Interleukin 2”, ed. K. A. Smith, Academic Press, Inc., San Diego, Calif., p237 (1988); Rosenberg, S., Ann. Surgery 208:121 (1988)). In fact, IL-2 has been utilized to treat subjects suffering from malignant melanoma, renal cell carcinoma, and acute myelogenous leukemia. (Rosenberg, S. A., et al., N. Eng. J. Med. 316:889-897 (1978); Bukowski, R. M., et al., J. Clin. Oncol 7:477-485 (1989); Foa, R., et al., Br. J. Haematol. 77:491-496 (1990)).

Another cytokine with promise as an anticancer and antiviral agent is interferon-α. Interferon-α (IFN-α) is an IFN type I cytokine, has been employed to treat leukemia, myeloma, and renal cell carcinomas. IFN type I cytokines have been shown to increase class I MHC molecule expression. Because most cytolytic T-cells (CTLs) recognize foreign antigens bound to class I MHC molecules, type I IFNs may boost the effector phase of cell-mediated immune responses by enhancing the efficiency of CTL-mediated killing. At the same time, type I IFN may inhibit the cognitive phase of immune responses, by preventing the activation of class II MHC-restricted helper T-cells. IL-12, IL-15, and various flavonoids can also increase the T-cell response.

In Vivo Results of Histamine Agonist Treatments

Histamine is a biogenic amine, i.e. an amino acid that possesses biological activity mediated by pharmacological receptors after decarboxylation. The role of histamine in immediate type hypersensitivity is well established. (Plaut, M. and Lichtenstein, L. M. 1982 Histamine and immune responses. In Pharmacology of Histamine Receptors, Ganellin, C. R. and M. E. Parsons eds. John Wright & Sons, Bristol pp. 392-435.)

Examinations of whether a H₂-receptor agonists or antagonists can be applied to the treatment of cancer have yielded contradictory results. Some reports suggest that administration of histamine alone suppressed tumor growth in hosts having a malignancy. (Burtin, Cancer Lett. 12:195 (1981)). On the other hand, histamine has been reported to accelerate tumor growth in rodents. (Nordlund, J. J., et al., J. Invest. Dermatol 81:28 (1983)).

Similarly, contradictory results were obtained when the effects of histamine-receptor antagonists were evaluated. Some studies report that histamine-receptor antagonists suppress tumor development in rodents and humans. (Osband, M. E., et al., Lancet 1 (8221):636 (1981)). Other studies report that such treatment enhances tumor growth and may even induce tumors. (Barna, B. P., et al., Oncology 40:43 (1983)).

Synergistic Effects of a H₂-Receptor Agonist and IL-2

Despite the conflicting results when histamine is administered alone, recent reports clearly reveal that histamine acts synergistically with cytokines to augment the cytotoxicity of NK cells. For example, studies using histamine analogues suggest that histamine's synergistic effects are exerted through the H₂-receptors expressed on the cell surface of monocytes. (Hellstrand, K., et al., J. Immunol. 137:656 (1986)).

Histamine's synergistic effect when combined with cytokines appears to result from the suppression of a down regulation of cytotoxicity mediated by other cell types present along with the cytotoxic cells. In vitro studies with NK cells alone confirm that cytotoxicity is stimulated when IL-2 is administered. However, in the presence of monocytes, the IL-2 induced enhancement of cytotoxicity of NK cells is suppressed. (See, U.S. Pat. No. 5,348,739, which is incorporated herein by reference).

In the absence of monocytes, histamine had no effect or weakly suppressed NK mediated cytotoxicity. (Hellstrand, K., et al., J. Immunol. 137:656 (1986); Hellstrand, K. and Hermodsson, S., Int. Arch. Allergy Appl. Immunol. 92:379-389 (1990)). Yet, NK cells exposed to histamine and IL-2 in the presence of monocytes exhibit elevated levels of cytotoxicity relative to that obtained when NK cells are exposed only to IL-2 in the presence of monocytes. Id. Thus, the synergistic enhancement of NK cell cytotoxicity by combined histamine and interleukin-2 treatment results not from the direct action of histamine on NK cells but rather from suppression of an inhibitory signal generated by monocytes.

Granulocytes have also been shown to suppress IL-2 induced NK-cell cytotoxicity in vitro. It appears that the H₂-receptor is involved in transducing histamine's synergistic effects on overcoming granulocyte mediated suppression. For example, the effect of histamine on granulocyte mediated suppression of antibody dependent cytotoxicity of NK cells was blocked by the H₂-receptor antagonist ranitidine and mimicked by the H₂-receptor agonist dimaprit. In contrast to the complete or nearly complete abrogation of monocyte mediated NK cell suppression by histamine and IL-2, such treatment only partially removed granulocyte mediated NK cell suppression. (U.S. Pat. No. 5,348,739; Hellstrand, K., et al., Histaminergic regulation of antibody dependent cellular cytotoxicity of granulocytes, monocytes and natural killer cells., J. Leukoc. Biol 55:392-397 (1994)).

As suggested by the experiments above, therapies employing histamine and cytokines are effective anticancer and antiviral strategies. U.S. Pat. No. 5,348,739 discloses that mice given histamine and IL-2 prior to inoculation with melanoma cell lines were protected against the development of lung metastatic foci. It has also been shown that a single dose of histamine could prolong survival time in animals inoculated intravenously with herpes simplex virus (HSV), and a synergistic effect on the survival time of animals treated with a combination of histamine and IL-2 was observed (Hellstrand, K., et al., Role of histamine in natural killer cell-dependent protection against herpes simplex virus type 2 infection in mice., Clin. Diagn. Lab. Immunol. 2:277-280 (1995)).

The above results demonstrate that strategies employing a combination of histamine and IL-2 are an effective means of treating malignancies and viral infection.

Presently the therapeutic potential of several immune cell stimulating compounds that show promise as efficacious anticancer and antiviral agents is diminished due to negatively regulating systems of the immune system. Accordingly, there is a need for methods which maximize the therapeutic potential of immune cell stimulating compounds.

### SUMMARY OF THE INVENTION

The teachings herein relate to methods for protecting cytotoxic T lymphocytes and NK cells in a subject, for the treatment of tumors, viral diseases or inflammatory diseases, comprising identifying a subject in need of cytotoxic T lymphocyte and NK cell protection; administering to the subject, an amount of diphenyliodonium (DPD, or other NADPH-oxidase inhibitor, effective to protect cytotoxic T lymphocytes and NK cells in the presence of monocytes or macrophages. In certain embodiments, the amount of DPI is selected from a daily dose between about 10 to 100 mg/kg. In more particular embodiments, the amount of DPI is administered at a daily dose of about 10 mg/kg, about 50 mg/kg, or about 100 mg/kg.

In further embodiments, the methods herein can also include administering an effective amount of a cytotoxic lymphocyte stimulatory composition to the subject, wherein said cytotoxic lymphocyte stimulatory composition is selected from the group consisting of a vaccine adjuvant, a vaccine, a peptide, a cytokine, and a flavonoid.

In particular embodiments, the cytotoxic lymphocyte stimulatory composition is a cytokine selected from the group consisting of IL-1, IL-2, IL-12, IL-15, IFN-α, IFN-β, and IFN-γ. It can also be advantageous to use a flavonoid selected from the group consisting of flavone acetic acids and xanthenone-4-acetic acids.

The cytotoxic lymphocyte stimulatory composition is administered in a daily dose of between about 1,000 and about 600,000 U/kg.

The methods herein can further comprise administering an effective amount of a compound that inhibits the production or release of intercellular reactive oxygen metabolites (ROM) selected from the group consisting of histamine, histamine dihydrochloride, histamine phosphate, serotonin, dimaprit, clonidine, tolazoline, impromadine, 4-methylhistamine, betazole, and a histamine congener.

These compounds can be effectively administered between about 0.05 and about 50 mg per dose. In further embodiments, these compounds can be administered between about 1 and about 500 μg/kg of patient weight per dose. In specific embodiments, the administration of DPI and a compound that inhibits the production or release of intercellular reactive oxygen metabolites (ROM) is performed within 1 hour, or at least within 24 hours. In certain embodiments, the intercellular reactive oxygen metabolite is hydrogen peroxide. In other embodiments, the ROM is superoxide, nitric oxide or the hydroxyl radical.

Methods herein can also comprise administering an effective amount of a ROM scavenger, such as a scavenger of intercellular hydrogen peroxide. These scavengers can include catalase, glutathione peroxidase, superoxide dismutase (SOD), vitamin E, vitamin A, vitamin C, SOD mimetics, ascorbate peroxidase, and the like. In certain embodiments, the ROM scavenger is administered in a dose of from about 0.05 to about 50 mg/day.

In some embodiments, an effective amount of DPI, a compound that inhibits NADPH-oxidase, which leads to inhibition of the production or release of intercellular reactive oxygen metabolites, and a scavenger of hydrogen peroxide are administered separately.

The methods herein can further comprise administering a chemotherapeutic agent. Chemotherapeutic agents can comprise an anticancer agent selected from the group consisting of cyclophosphamide, chlorambucil, melphalan, estramustine, iphosphamide, prednimustin, busulphan, tiottepa, carmustin, lomustine, methotrexate, azathioprine, mercaptopurine, thioguanine, cytarabine, fluorouracil, vinblastine, vincristine, vindesine, etoposide, teniposide, dactinomucin, doxorubin, dunorubicine, epirubicine, bleomycin, nitomycin, cisplatin, carboplatin, procarbazine, amacrine, mitoxantron, tamoxifen, nilutamid, and aminoglutemide, for example.

In specific embodiments, the effective administration of DPI, or another NADPH-oxidase inhibitor, and the chemotherapeutic agent are performed concomitantly.

Further embodiments include compositions to protect cytotoxic T lymphocytes and NK cells in a subject, for the treatment of tumors, viral diseases or inflammatory diseases, comprising an effective amount of NADPH-oxidase inhibitor, such as diphenyliodonium (DPI), in a pharmaceutically acceptable carrier. Compositions can further comprise a cytotoxic lymphocyte stimulatory compound selected from the group consisting of a vaccine adjuvant, a vaccine, a peptide, a cytokine, and a flavonoid. Specific cytokines can include IL-1, IL-2, IL-12, IL-15, IFN-α, IFN-β, and IFN-γ.

Examples of flavonoids include flavone acetic acids and xanthenone-4-acetic acids. In certain embodiments, cytotoxic lymphocyte stimulatory compositions can be administered in a daily dose of between about 1,000 and about 600,000 U/kg.

Compositions provided herein can further comprise an effective amount of a compound that inhibits the production or release of intercellular reactive oxygen metabolites (ROM) selected from the group consisting of histamine, histamine dihydrochloride, histamine phosphate, serotonin, dimaprit, clonidine, tolazoline, impromadine, 4-methylhistamine, betazole, and a histamine congener. These compounds can be administered between about 0.05 and about 50 mg per dose, or between about 1 and about 500 μg/kg of patient weight, for example.

Compositions herein can further comprise a chemotherapeutic agent. More specifically, chemotherapeutic agents can include an anticancer agent selected from the group consisting of cyclophosphamide, chlorambucil, melphalan, estramustine, iphosphamide, prednimustin, busulphan, tiottepa, carmustin, lomustine, methotrexate, azathioprine, mercaptopurine, thioguanine, cytarabine, fluorouracil, vinblastine, vincristine, vindesine, etoposide, teniposide, dactinomucin, doxorubin, dunorubicine, epirubicine, bleomycin, nitomycin, cisplatin, carboplatin, procarbazine, amacrine, mitoxantron, tamoxifen, nilutamid, and aminoglutemide.

Compositions described herein can include an effective amount of DPI administered between about 10 to about 100 mg/kg. More specifically about 10 mg/kg, about 50 mg/kg, or about 100 mg/kg.

The teachings herein also relate to methods and compositions for facilitating activation and protection of cytotoxic lymphocytes. In one embodiment, the teachings herein relate to a method comprising identifying a patient in need of enhanced cytotoxic lymphocyte activity, and administering to the patient an amount of an NADPH-oxidase inhibitor, such as diphenyliodonium (DPI), effective to activate and protect cytotoxic lymphocyte function in the presence of MO.

In another embodiment, the methods herein further comprise administering a cytotoxic lymphocyte stimulatory composition. In various aspects of this embodiment, the composition may be a vaccine adjuvant, a vaccine, a peptide, a cytokine or a flavonoid. Vaccine adjuvants for use with the teachings herein may be selected from the group consisting of bacillus Calmette-Guerin (BCG), pertussis toxin (PT), cholera toxin (CT), E. coli heat-labile toxin (LT), mycobacterial 71-kDa cell wall associated protein, microemulsion MF59, microparticles of poly(lactide-co-glycolides)(PLG), and immune stimulating complexes (ISCOMS). Vaccines for use with the teachings herein may be selected from the group consisting of influenza vaccines, human immunodeficiency virus vaccines, Salmonella enteritidis vaccines, hepatitis B vaccines, Boretella bronchiseptica vaccines, tuberculosis vaccines, allogeneic cancer vaccines, and autologous cancer vaccines. The teachings herein contemplate the use of a variety of cytokines and flavonoids. The cytokines may be selected from IL-1, IL-2, IL-12, IL-15, IFN-α, IFN-β, or IFN-γ, for example. Flavonoids may be selected from the group consisting of flavone acetic acids and xanthenone-4-acetic acids, for example. Cytotoxic lymphocyte stimulatory compositions can be administered in a daily dose to an adult human of between about 1,000 and about 600,000 U/kg, for example.

Another embodiment of the teachings herein contemplates the use of compounds effective to inhibit the production or release of intercellular hydrogen peroxide selected from the group consisting of histamine, histamine phosphate, serotonin, dimaprit, clonidine, tolazoline, impromadine, 4-methylhistamine, betazole, and a histamine congener. In one aspect of this embodiment, these compounds may be administered to an adult human at between about 0.05 and about 50 mg per dose. In another aspect of this embodiment, these compounds may also be administered at between about 1 and about 500 μg/kg of patient weight per dose.

Another embodiment of the teachings herein contemplates administration of the cytotoxic lymphocyte activating compound and the ROM inhibitory compound within 1 hour of each other. Another embodiment contemplates administration of the cytotoxic lymphocyte activating and protecting compound and the ROM inhibitory compound within 24 hours of each other.

The methods herein further contemplate an embodiment in which an effective amount of a scavenger of ROM like intercellular hydrogen peroxide is administered. In one aspect of this embodiment, the scavenger may be selected from the group consisting of catalase, glutathione peroxidase, superoxide dismutase (SOD), vitamin E, vitamin A, vitamin C, SOD mimetics, ascorbate peroxidase, and the like. In another aspect of this embodiment, the hydrogen peroxide scavenger may be administered to an adult human in a dose of from about 0.05 to about 50 mg/day and the compounds maybe administered together or separately.

In addition to the compounds discussed above, the teachings herein contemplate the administration of a variety of chemotherapeutic agents. In one embodiment, the chemotherapeutic agent is an anticancer agent selected from the group consisting of cyclophosphamide, chlorambucil, melphalan, estramustine, iphosphamide, prednimustin, busulphan, tiottepa, carmustin, lomustine, methotrexate, azathioprine, mercaptopurine, thioguanine, cytarabine, fluorouracil, vinblastine, vincristine, vindesine, etoposide, teniposide, dactinomucin, doxorubin, epirubicine, bleomycin, nitomycin, cisplatin, carboplatin, procarbazine, amacrine, mitoxantron, tamoxifen, nilutamid, and aminoglutemide. Conventional dosages of these agents can be used. In another embodiment, the chemotherapeutic agent administered is an antiviral agent, selected from the group consisting of idoxuridine, trifluorothymidine, adenine arabinoside, acycloguanosine, bromovinyldeoxyuridine, ribavirin, trisodium phosphophonoformate, amantadine, rimantadine, (S)-9-(2,3-Dihydroxypropyl)-adenine, 4′,6-dichloroflavan, AZT, 3′(-azido-3′-deoxythymidine), ganciclovir, didanosine (2′,3′-dideoxyinosine or ddI), zalcitabine (2′,3′-dideoxycytidine or ddC), dideoxyadenosine (ddA), nevirapine, inhibitors of the HIV protease, and other viral protease inhibitors. Conventional dosages of these agents can be used.

### BRIEF DESCRIPTION OF THE DRAWINGS

FIG. 1 is a graph showing the protection of CD3ε+ T-cells against oxidative inhibition by DPI. Lymphocytes and MO were recovered from peripheral blood as described herein. A mixture of MO and lymphocytes was treated with culture medium (control, open circles) or IL-2 (100 U/ml; filled circles) for 16 hrs. After incubation, lymphocytes were labeled with antibodies to CD3ε and CD69. Data show CD69 expression in viable T-cells (CD3ε+) (left panel), and the percentage of T-cells with reduced forward and increased side angle scatter characteristic of apoptosis (right panel). Similar results were obtained when CD69 expression was examined in CD56+. NK-cells incubated with MO: 29.5% (control) or 79.0% (DPI 1000 nM) of NK-cells acquired the CD69 antigen in response to IL-2. DPI did not increase the IL-2-induced expression of CD69 in T-cells or NK-cells incubated in the absence of MO (not shown). The results are representative of three similar experiments.

### DETAILED DESCRIPTION OF THE INVENTION

The teachings herein relate to methods of treating conditions such as cancer, viral diseases, and inflammatory diseases with a ROM inhibitory compound that is administered alone or in conjunction with additional agents. A ROM inhibitory compound is any compound of composition that inhibits the production and/or release of ROM. The administration of these various agents results in the activation and protection of cytotoxic lymphocytes from the deleterious and inhibitory effects of monocytes/macrophages, as well as a stimulation of the anti-cancer and anti-viral properties of these cells. In addition, the administration of a ROM inhibitory compound in the presence of a vaccine composition results in an increase in lymphocyte proliferation in the presence of monocytes. The addition of other agents that are cytotoxic lymphocyte activation compounds is also contemplated. Cytotoxic lymphocytes are lymphocyte that possess cytotoxic capabilities such as NK-cells and cytotoxic T-cells (CTLs). The term cytotoxic lymphocytes also encompasses non-cytotoxic cells such as T-helper cells that assist in the activation of a lymphocyte with cytotoxic capabilities. Cytotoxic lymphocyte activation compounds, including those that have an immunological stimulatory character, preferably function in a synergistic fashion with a ROM inhibitory compound. Representatives of such immunological stimulatory compounds include cytokines, peptides, flavonoids, antigens generally, vaccines, and vaccine adjuvants. Additional classes of agents usable with the methods disclosed herein encompass chemotherapeutic and/or antiviral agents. These methods are useful for treating neoplastic as well as viral disease.

In contemplating the treatment of individuals suffering from various neoplastic and viral diseases, the teachings herein seek to stimulate and enhance cell-mediated immunity to accomplish that end. Cell-mediated immunity (CMI) comprises the cytotoxic lymphocyte-mediated immune response to a “foreign agent.” The CMI response differs from the antibody-mediated humoral immunity in that the active agent in CMI is a cytotoxic lymphocyte rather than an antibody protein.

Cell-mediated immunity (CMI) operates with cytotoxic lymphocytes such as NK-cells and/or T-cells (CTLs) recognizing and destroying cells displaying “foreign” antigens on their surface. In the teachings herein, a foreign agent may be a neoplastic cell or a virus infected cell. As such, CMI functions to eliminate foreign cells from the body. For example, CMI would target cells infected with a virus, rather than to prevent the infection of the cell. Cell-mediated immunity, unlike humoral immunity which can be effective to prevent viral infection, remains the principal mechanism of defense against established viral infections. It is also pivotal in combating neoplastic disease. Therefore, the cytotoxic lymphocyte activity enhancing aspects of the teachings herein are uniquely suited to combat neoplastic and viral diseases.

As discussed above, the immune system contains a number of different cell types, each of which serve to protect the body from foreign invasion. Certain cells of the immune system produce reactive oxygen metabolite (ROM) such as hydrogen peroxide, hypohalous acids, and hydroxyl radicals to achieve this goal. In previous observations, activation of human natural killer (NK)-cells, a type of cytotoxic lymphocytes, in response to in vitro cytokine stimulation (e.g., IL-2 or IFN-α) is effectively inhibited by autologous monocytes/macrophages (MO). (For review see, Hellstrand, K., et al., Scand. J. Clin. Lab Invest. 57:193-202 (1997)). The inhibitory signal is conveyed by hydrogen peroxide or other reactive oxygen metabolites (ROM) generated by MO. (See Hellstrand, K., et al., J. Immunol., 153: 4940-4947 (1994); Hansson, M., et al., J. Immunol. 156:42-47 (1996)). Addition of scavengers which reduce the concentration of ROM and/or the addition of compounds which inhibit the release of ROM, such as histamine or H₂-receptor agonists, both have been shown to remove the inhibitory effects of MO. Id.

T-cells are considered important effector cells responsible for the antitumor properties of various cytokines such as IFN-α and IL-2, observed in experimental tumor models and in human neoplastic disease. (Sabzevari, H., et al., Cancer Res. 53: 4933-4937, (1993); Hakansson, A., et al., Br. J. Cancer, 74: 670-676, (1996); Wersall and Mellstedt, Med. Oncol., 12: 69-77, (1995)). The teachings herein relate, in part, to methods where compounds which reduce the concentration of ROM are used in conjunction with one or more T-cell activation compounds that result in T-cell activation or stimulation. The teachings herein, through the administration of ROM affecting compounds, T-cell activating compounds, and/or anticancer and antiviral compounds, provide methods to treat neoplastic disorders as well as viral infections by increasing the number and specific activity of T-cells.

A number of cytotoxic lymphocyte activation compounds are known in the art to activate and stimulate cytotoxic lymphocyte activity. The dosing, routes of administration and protocols for the use and administration of these materials can be the conventional ones, well known in the art. Generally, interleukins, cytokines and flavonoids have been shown to stimulate cytotoxic lymphocyte activity. Examples of suitable compounds are selected from the group consisting of IL-1, IL-2, IL-12, IL-15, IFN-α, IFN-β, TFN-γ and flavone acetic acid, xanthenone-4-acetic acid, and analogues or derivatives thereto.

Certain vaccines and vaccine adjuvants may also be considered cytotoxic lymphocyte activating compounds. Compounds contemplated here include a number of vaccines and vaccine adjuvants that assist administered antigens to induce rapid, potent, and long-lasting cytotoxic lymphocyte-mediated immune responses, from immunized or vaccinated individuals. Illustrative vaccines include influenza vaccines, human immunodeficiency virus vaccines, Salmonella enteritidis vaccines, hepatitis B vaccines, Boretella bronchiseptica vaccines, and tuberculosis vaccines, as well as various anticancer therapeutic vaccines such as allogeneic cancer and autologous cancer vaccines which are known in the art.

One aspect of the teachings herein is directed toward the use of a variety of vaccine adjuvants. Such agents including bacillus Calmette-Guerin (BCG), pertussis toxin (PT), cholera toxin (CT), E. coli heat-labile toxin (LT), mycobacterial 71-kDa cell wall associated protein, the vaccine adjuvant oil-in-water microemulsion MF59, microparticles prepared from the biodegradable polymers poly(lactide-co-glycolides) (PLG), immune stimulating complexes (iscoms) which are 30-40 nm cage-like structures, (which consist of glycoside molecules of the adjuvant Quil A, cholesterol and phospholipids in which antigen can be integrated), as well as other suitable compounds and compositions known in the art. Such compounds may be administered in amounts sufficient to elicit an effective immune response from an immunized individual.

The teachings herein contemplate and disclose a number of different cytotoxic lymphocyte activating compounds. These compounds may be used to form cytotoxic lymphocyte activating compositions that may be administered as a step of the methods herein to achieve the activation of a patient's cytotoxic lymphocytes. The teachings herein contemplate the use of the terms cytotoxic lymphocyte activating compound and cytotoxic lymphocyte activation compositions as interchangeable. The dosing, routes of administration and protocols for the use and administration of these materials can be the conventional ones, well known in the art.

The term “reactive oxygen metabolite inhibitors” encompasses a number of disparate compounds. NADPH inhibitors, H₂-receptor agonists, and other compounds with H₂-receptor agonist activity, suitable for use in the teachings herein, are known in the art. Examples of suitable compounds include diphenyliodonium (DPI), histamine, compounds with a chemical structure resembling that of histamine or serotonin, yet do not negatively affect H₂-receptor activities. Suitable compounds include, but are not limited to, DPI, histamine, dimaprit, clonidine, tolazoline, impromadine, 4-methylhistamine, betazole, histamine congeners, H₂-receptor agonists, 8-OH-DPAT, ALK-3, BMY 7378, NAN 190, lisuride, d-LSD, flesoxinan, DHE, MDL 72832, 5-CT, DP-5-CT, ipsapirone, WB 4101, ergotamine, buspirone, metergoline, spiroxatrine, PAPP, SDZ (−) 21009, and butotenine.

A variety of ROM scavengers, including hydrogen peroxide (H₂O₂) scavengers, effective to catalyze the decomposition of intercellular H₂O₂ are also known in the art. Suitable compounds include, but are not limited to, catalase, glutathione peroxidase, vitamin E, vitamin A, vitamin C, SOD, SOD mimetics, ascorbate peroxidase, and the like.

Administration of the compounds discussed above can be practiced in vitro or in vivo. When practiced in vitro, any sterile, non-toxic route of administration may be used. When practiced in vivo, administration of the compounds discussed above may be achieved advantageously by subcutaneous, intravenous, intramuscular, intraocular, oral, transmucosal, or transdermal routes, for example by injection or by means of a controlled release mechanism. Examples of controlled release mechanisms include polymers, gels, microspheres, liposomes, tablets, capsules, suppositories, pumps, syringes, ocular inserts, transdermal formulations, lotions, creams, transnasal sprays, hydrophilic gums, microcapsules, inhalants, and colloidal drug delivery systems.

The compounds are administered in a pharmaceutically acceptable form and in substantially non-toxic quantities. A variety of forms of the compounds administered are contemplated by the teachings herein. The compounds may be administered in water with or without a surfactant such as hydroxypropyl cellulose. Dispersions are also contemplated, such as those utilizing glycerol, liquid polyethylene glycols, and oils. Antimicrobial compounds may also be added to the preparations. Injectable preparations may include sterile aqueous solutions or dispersions and powders which may be diluted or suspended in a sterile environment prior to use. Carriers such as solvents or dispersion media contain water, ethanol polyols, vegetable oils and the like may also be added to the compounds provided herein. Coatings such as lecithins and surfactants may be used to maintain the proper fluidity of the composition. Isotonic agents, such as sugars or sodium chloride, may be added, as well as products intended to delay absorption of the active compounds such as aluminum monostearate and gelatin. Sterile injectable solutions are prepared according to methods well known to those of skill in the art and can be filtered prior to storage and/or use. Sterile powders may be vacuum or freeze dried from a solution or suspension. Sustained-release preparations and formulations are also contemplated by the teachings herein. Any material used in the compositions described herein should be pharmaceutically acceptable and substantially non-toxic in the amounts employed.

Although in some of the experiments that follow the compounds are used at a single concentration, it should be understood that in the clinical setting, the compounds may be administered in multiple doses over prolonged periods of time. Typically, the compounds may be administered for periods up to about one week, and even for extended periods longer than one month or one year. In some instances, administration of the compounds may be discontinued and then resumed at a later time. A daily dose of the compounds may be administered in several doses, or it may be given as a single dose.

In addition, the compounds can be administered separately or as a single composition (combined). If administered separately, the compounds should be given in a temporally proximate manner, e.g., within a twenty-four hour period, such that the activation of cytotoxic lymphocytes by the cytokine or other compound is enhanced. More particularly, the compounds may be given within 1 hour of each other. The administration can be by either local or by systemic injection or infusion. Other methods of administration may also be suitable.

The teachings herein also contemplate combinations of cytotoxic lymphocytes activation compounds with ROM production or release inhibiting compounds, ROM scavenging compounds, anticancer compounds, and combinations of antiviral compounds. The doses, routes of administration, and protocols for the use and administration of these materials can be the conventional ones, well known in the art. For example, in one embodiment, IL-2 and IL-12 are combined to activate a population of cytotoxic lymphocytes. In an alternative embodiment, a vaccine or an adjuvant could be used to activate a population of T-cells. In another embodiment, DPI is combined with histamine to inhibit the production or release of ROM from monocytes during a treatment regime. Combinations of other compounds, including ROM scavengers such as catalase, glutathione peroxidase, vitamin E, vitamin A, vitamin C, SOD, SOD mimetics, ascorbate peroxidase, for example, are also contemplated. The teachings herein further contemplate using combinations of all of the various compounds discussed above to prepare an effective means to stimulate cytotoxic lymphocytes against neoplastic and/or viral disease.

All compound preparations are provided in dosage unit forms for uniform dosage and ease of administration. Each dosage unit form contains a predetermined quantity of active ingredient calculated to produce a desired effect in association with an amount of pharmaceutically acceptable carrier. Such a dosage would therefore define an effective amount of a particular compound.

A preferred compound dosage range can be determined using techniques known to those having ordinary skill in the art. IL-2, IL-12 or IL-15 can be administered in an amount of from about 1,000 to about 600,000 U/kg/day (18 MIU/m²/day or 1 mg/m²/day); more preferably, the amount is from about 3,000 to about 200,000 U/kg/day, and even more preferably, the amount is from about 5,000 to about 10,000 U/kg/day.

IFN-α, IFN-β, and IFN-γ can also be administered in an amount of from about 1,000 to about 600,000 U/kg/day; more preferably, the amount is from about 3,000 to about 200,000 U/kg/day, and even more preferably, the amount is from about 10,000 to about 100,000 U/kg/day.

Flavonoid compounds can be administered in an amount of from about 1 to about 100,000 mg/day; more preferable, the amount is from about 5 to about 10,000 mg/day, and even more preferably, the amount is from about 50 to about 1,000 mg/day.

Commonly used doses for the compounds described herein fall within the ranges listed herein. For example, L-2 is commonly used alone in doses of about 300,000 U/kg/day. IFN-α is commonly used at 45,000 U/kg/day. IL-12 has been used in clinical trials at doses of 0.5-1.5 μg/kg/day. Motzer, et al., Clin. Cancer Res. 4(5):1183-1191 (1998). IL-1 beta has been used at 0.005 to 0.2 μg/kg/day in cancer patients. Triozzi, et al., J. Clin. Oncol. 13(2):482-489 (1995). IL-15 has been used in rates in doses of 25-400 μg/kg/day. Cao, et al., Cancer Res 58(8):1695-1699 (1998).

Vaccines and vaccine adjuvants can be administered in amounts appropriate to those individual compounds to activate cytotoxic lymphocytes. Appropriate doses for each can readily be determined by techniques well known to those of ordinary skill in the art. Such a determination will be based, in part, on the tolerability and efficacy of a particular dose using techniques similar to those used to determine proper chemotherapeutic doses.

Compounds effective to inhibit the release or formation of intercellular hydrogen peroxide, or scavengers of hydrogen peroxide, can be administered in an effective amount from about 0.05 to about 10 mg/day; more preferable, the amount is from about 0.1 to about 8 mg/day, and even more preferably, the amount is from about 0.5 to about 5 mg/day. Alternatively, these compounds may be administered from 1 to 100 micrograms per kilogram of patient body weight (1 to 100 μg/kg). However, in each case, the dose depends on the activity of the administered compound. The foregoing doses are appropriate and effective for inhibitors such as DPI, histamine, H₂-receptor agonists, other intercellular ROM production or release inhibitors or ROM scavengers. Appropriate doses for any particular host can be readily determined by empirical techniques well known to those of ordinary skill in the art.

The teachings herein contemplate identifying a patient in need of enhanced cytotoxic lymphocyte activity and increasing that patient's circulating blood ROM inhibitory compound concentration to an optimum, beneficial, therapeutic level so as to provide for more efficient cytotoxic lymphocyte stimulation. Such a level may be achieved through repeated injections of the compounds described herein in the course of a day, during a period of treatment.

Subjects suffering from cancer often exhibit decreased levels of circulating blood histamine. (Burtin et al. Decreased blood histamine levels in subjects with solid malignant tumors, Br. J. Cancer 47: 367-372 (1983)). Thus, the elevation of blood histamine concentrations to beneficial levels finds ready application to cancer and antiviral treatments based on synergistic effects between histamine and agents which enhance cytotoxic effector cell mediated cytotoxicity. In such protocols, the activity of T-cells is enhanced. For example, the cytotoxic activity of cytotoxic T lymphocytes (CTLs) is enhanced by combining the administration of a H₂-receptor agonist such as histamine to increase circulating histamine to a beneficial level sufficient to augment the activity of an agent which acts in synergy with a H₂-receptor agonist to increase cytotoxicity with the administration of the agent.

In one embodiment, beneficial levels of circulating blood ROM inhibitory compound levels, such as DPI or H₂-receptor agonist, are obtained by administering the ROM inhibitory compound at a dosage of 0.05 to 10 mg/day. In another embodiment, beneficial blood levels of ROM inhibitory compounds are administered at 1 to 100 micrograms per kilogram of patient body weight (1 to 100 μg/kg).

In still other embodiments, the ROM inhibitory compounds are administered at 1 to 100 milligrams per kilogram of patient body weight (1 to 100 mg/kg). For example the ROM inhibitor can be administered at 1 mg/kg, 10 mg/kg, 50 mg/kg, or 100 mg/kg, for example.

In another embodiment, the ROM inhibitory compound is administered over a treatment period of 1 to 4 weeks with injections occurring as frequently as several times daily, over a period of up to 52 weeks. In one embodiment, the ROM inhibitory compound can be administered for 9 days. In still another embodiment, the ROM inhibitory compound is administered for a period of 1-2 weeks, with multiple injections occurring as frequently as several times daily. This administration can be repeated every few weeks over a time period of up to 52 weeks, or longer. Additionally, the frequency of administration may be varied depending on the patient's tolerance of the treatment and the success of the treatment. For example, the administrations may occur three times per week, or even daily, for a period of up to 24 months.

Further embodiments contemplate utility with respect to the treatment of various cancers or neoplastic diseases. Malignancies against which the teachings herein may be directed include, but are not limited to, primary and metastatic malignant tumor disease, hematological malignancies such as acute and chronic myelogenous leukemia, acute and chronic lymphatic leukemia, multiple myeloma, Waldenstroms Macroglobulinemia, hairy cell leukemia, myelodysplastic syndrome, polycytaemia vera, and essential thrombocytosis. In more specific embodiments, an ROM inhibitor, such as DPI is administered to a subject, in order to inhibit the growth of a tumor.

The methods described herein can also be utilized alone or in combination with other anticancer therapies. When used in combination with a chemotherapeutic regime, a ROM inhibitory compound and a cytotoxic lymphocyte activating compound are administered with a chemotherapeutic agent or agents. The doses, routes of administration and protocols for the use and administration of these materials can be the conventional ones, well known in the art. Representative compounds used in cancer therapy include cyclophosphamide, chlorambucil, melphalan, estramustine, iphosphamide, prednimustin, busulphan, tiottepa, carmustin, lomustine, methotrexate, azathioprine, mercaptopurine, thioguanine, cytarabine, fluorouracil, vinblastine, vincristine, vindesine, etoposide, teniposide, dactinomucin, doxorubin, dunorubicine, epirubicine, bleomycin, nitomycin, cisplatin, carboplatin, procarbazine, amacrine, mitoxantron, tamoxifen, nilutamid, and aminoglutemide. Procedures for employing these compounds against malignancies are well established. In addition, other cancer therapy compounds may also be utilized.

The teachings herein also contemplate treatment of a variety of viral diseases. The following are merely examples of some of the viral diseases against which the teachings herein are effective. There are a number of herpetic diseases caused by herpes simplex or herpes zoster viruses including herpes facialis, herpes genitalis, herpes labialis, herpes praeputialis, herpes progenitalis, herpes menstrualis, herpetic keratitis, herpes encephalitis, herpes zoster ophthalmicus, and shingles.

In another aspect, the teachings herein are effective against viruses that cause diseases of the enteric tract, such as rotavirus mediated disease. In still other aspects, the teachings herein are effective against various blood based infections, such as: yellow fever, dengue, ebola, Crimean-Congo hemorrhagic fever, hanta virus disease, mononucleosis, and HIV/AIDS.

Another aspect of the teachings herein is directed toward various hepatitis causing viruses. A representative group of these viruses includes: hepatitis A virus, hepatitis B virus, hepatitis C virus, hepatitis D virus, and hepatitis E virus.

In still another aspect, the teachings herein are effective against respiratory tract diseases caused by viral infections, such as: rhinovirus infection (common cold), mumps, rubella, varicella, influenza B, respiratory syncytial virus infection, measles, acute febrile pharyngitis, pharyngoconjunctival fever, and acute respiratory disease.

Another aspect of the teachings herein contemplates treatment for various cancer linked viruses, including: adult T-cell leukemia/lymphoma (HTLVs), nasopharyngeal carcinomas, Burkitt's lymphoma (EBV), cervical carcinomas, and hepatocellular carcinomas.

In still a further aspect, the teachings herein are useful in the treatment of viral-meditated encephalitis, including: St. Louis encephalitis, Western encephalitis, and tick-borne encephalitis.

The methods described herein can also be utilized alone or in combination with other antiviral therapies. When used in combination with an antiviral chemotherapeutic regime, a ROM inhibitory compound, and the cytotoxic lymphocyte activating compound are administered with an antiviral chemotherapeutic agent or agents. The doses, routes of administration and protocols for the use and administration of these materials can be the conventional ones, well known in the art. Representative compounds used in antiviral chemotherapy include idoxuridine, trifluorothymidine, adenine arabinoside, acycloguanosine, bromovinyldeoxyuridine, ribavirin, trisodium phosphophonoformate, amantadine, rimantadine, (S)-9-(2,3-Dihydroxypropyl)-adenine, 4′,6-dichloroflavan, AZT, 3′(-azido-3′-deoxythymidine), ganciclovir, didanosine (2′,3′-dideoxyinosine or ddI), zalcitabine (2′,3′-dideoxycytidine or ddC), dideoxyadenosine (ddA), nevirapine, inhibitors of the HIV protease, and other viral protease inhibitors.

The teachings herein also contemplate using a combination of anticancer and antiviral agents in conjunction with the administration of a ROM inhibitory compound.

Although not intended to be limiting, it is contemplated that the methods herein augment cytotoxic lymphocyte activity by altering the mechanics of antigen presentation. One theory provides that monocytes/macrophages that are also antigen presenting cells (APC) are inhibited from presenting antigens to T-cells. This inhibition might result from MO metabolic pathways dedicated to the generation of ROM that inhibit MO antigen presenting metabolic pathways, producing mutually exclusive antigen presenting or ROM producing states in MO populations. A result of the inhibition of MO antigen presentation is that T-cell populations would remain dormant in the absence of presented antigen and in the presence of ROM.

Under this theory, administration of ROM production and release inhibiting compounds, such as histamine, act to increase T-cell activity by increasing antigen presentation. Monocytes producing ROM may have a molecular switch thrown in the present of beneficial concentrations of histamine that results in a down regulation of ROM production and an increase in antigen presentation capacity. In the mutually exclusive metabolic state hypothesized above, the down regulation of ROM production results in a subsequent increase in antigen presentation pathways and thus antigen presentation. Accordingly, administration of histamine or other ROM inhibiting compounds in the presence of an antigen based T-cell activator, like a vaccine, would serve to increase T-cell activity by decreasing ROM production and increasing antigen presentation.

In an alternative theory, the administration of a ROM inhibitory compound, results in an increase cytotoxic lymphocyte activity by removing ROM induced cytotoxic lymphocyte inhibition.

The examples discussed below apply the teachings herein and show that monocytes/macrophages (MO), and particularly MO-derived reactive oxygen metabolites (ROMs), effectively suppress the activation of human cytotoxic lymphocytes even after the in vitro administration of cytotoxic lymphocyte activation compounds such IFN-α or IL-2. Furthermore, it is shown that the addition of a ROM inhibitory compound confers protection to cytotoxic lymphocytes when added to a mixture of lymphocytes and MO.

To determine the effect of the various compounds on a population of T-cells, the expression of various cytotoxic lymphocyte markers that are inducibly expressed on the surface of mature human cytotoxic lymphocytes was studied. The observed results show that cytokine-induced activation of cytotoxic lymphocytes, as reflected by the appearance of CD69 or other markers after incubation with representative cytokines such as IL-2 or IFN-α, was profoundly inhibited by MO in the absence of a ROM inhibitory compound. However, addition of such ROM inhibitory compounds effectively reversed the observed inhibitory effects of MO. Additional work was performed to study the effect of histamine on the proliferative response of human cytotoxic lymphocytes to a polyvalent vaccine against influenza virus in vitro. The administration of histamine and other ROM inhibiting compounds in these experiments was shown to elevate lymphocyte proliferation in presence of antigen and monocytes.

In further embodiments, the teachings herein can be used to treat inflammatory diseases. Examples of treatable inflammatory diseases include, COPD (chronic obstructive pulmonary disease), Rheumatoid Arthritis, Crohn's disease, lupus, septicaemia, meningitis, inflammatory bowel diseases and atheroscelrosis, for example. Inflammatory diseases that can be treated and/or prevented with the teachings herein are disclosed in U.S. application Ser. No. 10/171,018, filed Jun. 11, 2002, to Hellstrand et al., which is expressly incorporated herein by reference in its entirety. The NADPH oxidase inhibitors described herein, such as DPI, can be used to treat and/or prevent these diseases by inhibiting the release of ROM.

### EXAMPLES

Particular aspects herein can be more readily understood by reference to the following examples, which are intended to exemplify the teaching herein, without limiting their scope to the particular exemplified embodiments.

The methods herein may be used to enhance the activation and protection of cytotoxic lymphocyte populations using various cytotoxic lymphocyte activation compounds that result in cytotoxic lymphocyte stimulation and/or activation. ROM inhibitory compounds such as NADPH inhibitors, H₂-receptor agonists, and H₂O₂ scavengers and inhibitors are discussed below. To demonstrate the activation and protection characteristics of these compounds, lymphocytes (including NK-cells and T-cells) and monocytes were isolated from donated blood and examined for the activation characteristics when exposed various cytotoxic lymphocyte activating compounds, such as IL-2 and/or IFN-α, vaccines, vaccine adjuvants or other immunological stimulator compounds, various ROM inhibitory compounds, such as DPI, histamine, and various H₂O₂ scavengers, such as catalase.

Peripheral venous blood was obtained as freshly prepared leukopacks from healthy blood donors at the Blood Centre, Sahlgren's Hospital, Goteborg, Sweden, to study the activation characteristics of cytotoxic lymphocytes in the presence and absence of MO, and ROM inhibitors. The blood (65 ml) was mixed with 92.5 ml Iscove's medium, 35 ml 6% Dextran (Kabi Pharmacia, Stockholm, Sweden) and 7.5 ml acid citrate dextrose (ACD) (Baxter, Deerfield, Ill.). After incubation for 15 minutes at room temperature, the supernatant was carefully layered onto Ficoll-Hypaque (Lymphoprep, Myegaard, Norway). Mononuclear cells (MNC) were collected at the interface after centrifugation at 380 g for 15 minutes at room temperature, washed twice in PBS and resuspended in Iscove's medium supplemented with 10% human AB+serum. During all further separation of cells, the cell suspensions were kept in siliconized test tubes (Vacuette, Greiner, Stockholm).

The MNC were further separated into lymphocyte and monocyte (MO) populations using the counter-current centrifugal elutriation (CCE) technique originally described by Yasaka and co-workers (Yasaka, T. et al., J. Immunol., 127:1515) with modifications as described in Hansson, M., et al. (J. Immunol., 156: 42 (1996); hereby incorporated by reference). Briefly, the MNC were resuspended in elutration buffer containing 0.05% BSA and 0.015% EDTA in buffered NaCl and fed into a Beckman J2-21 ultracentrifuge with a JE-6B rotor at 2100 rpm. A fraction with >90% MO was obtained at a flow rate of 18 ml/min. A lymphocyte fraction enriched for NK-cells (CD3⁻/56⁺ phenotype) and T-cells (CD3⁺/56⁻) was recovered at flow rates of 14-15 ml/min. This fraction contained <3% MO and consisted of CD3,⁻/56⁺ NK-cells (45-50%), CD3,⁺/56⁻ T-cells (35-40%), CD3,⁻/56⁻ cells (5-10%), and CD3,⁺/56⁺ cells (1-5%), as judged by flow cytometry. In some experiments, dynabeads (Dynal A/S, Oslo, Norway) coated with anti-CD56 were used to obtain purified lymphocyte preparations of T-cells, as described in detail by Hansson, M., et al., incorporated above.

Following fractionation, the lymphocyte mixture of T-cells and NK cells was exposed to the various experimental conditions described below and assayed for activation using the appearance of certain cell surface proteins as indicia of activation.

Lymphocytes are identifiable by certain proteins which reside on the cell surface. Different cell surface proteins reside on different classes of lymphocytes and lymphocytes in different stages of activation. These proteins have been grouped into CD classes or “clusters of differentiation” and may serve as markers for different types of cells. Labeled antibodies, specific for different cell surface proteins, that bind to the different CD markers may be used to identify the different types of T-cells and their respective states of activation.

In the experiments described below, CD3, CD4, CD8, CD69 and CD56 (a NK-cell marker), were used to identify the cytotoxic lymphocytes of interest. The CD3 group of antibodies is specific for a marker expressed on all peripheral T-cells. The CD4 group of antibodies is specific for a marker on class II MHC-restricted T-cells, also known as T helper cells. The CD8 group of antibodies recognize a marker on class I MHC-restricted T-cells, also known as CTLs or cytolytic T-cells. The CD69 group of antibodies recognizes activated T-cells and other activated immune cells. Finally, the CD56 groups recognizes a heterodimer on the surface of NK-cells.

Flow cytometry was used in the experiments described below to identify the various sub-populations of T-cells. Flow cytometry permits an investigator to examine a population of cells using a number of labeled probes to differentiate sub-populations within the larger whole. In these experiments, the CD3 marker was used to identify the sub-population of T-cells and the CD4 and CD8 markers were used to further identify the sub-population of T-cells into T helper cells and CTLs. The effects of MO exposure in the presence and absence of histamine and T-cell activation compounds were determined using the CD69 T-cell activation marker. The expression of the different markers was estimated in a lymphocyte gate using flow cytometry (as described in Hellstrand, K., et al. Cell. Immunol. 138: 44-54 (1991), and hereby incorporated by reference).

The following protocol was used in experiments reporting the detection of surface antigens of cell populations. One million cells were incubated with appropriate fluorescein isothiocynate (FITC) and phycoerythrin (PE) conjugated monoclonal antibodies (Becton & Dickinson, Stockholm, Sweden; 1:1/10⁶ cells), on ice for 30 minutes. The cells were washed twice in PBS and resuspended in 500:1 sterile filtered PBS and analyzed by use of flow cytometry on a FACSort with a Lysys II software program (Becton & Dickenson). Lymphocytes were gated on the basis of forward and right angle scatter. The flow rate was adjusted to <200 cells×s⁻¹ and at least 5×10³ cells were analyzed for each sample, if not otherwise stated.

### Example 1

To assess whether DPI and histamine inhibited the spontaneous release of ROM from MO, a chemiluminescence assay was performed that specifically quantified extracellular ROM (superoxide anion). The spontaneous isoluminol-enhanced extracellular generation of superoxide anion in elutriated MO was measured using the assay described in Hellstrand, K., et al., J Immunol, 153:4940-4947 (1994) and in Lundqvist & Dahlgren, Free Radic. Biol. Mod. 20:785-792 (1996). A more than four fold reduction of released extracellular superoxide anion was observed at DPI concentrations of 10 nM, and similar results were obtained in three experiments using MO recovered from three blood donors. Similarly, histamine (at 50 μM) inhibited the concentration of extracellular superoxide anion in this model more than five-fold. This effect of histamine was completely antagonized by ranitidine, used at concentrations equimolar to histamine.

### Example 2

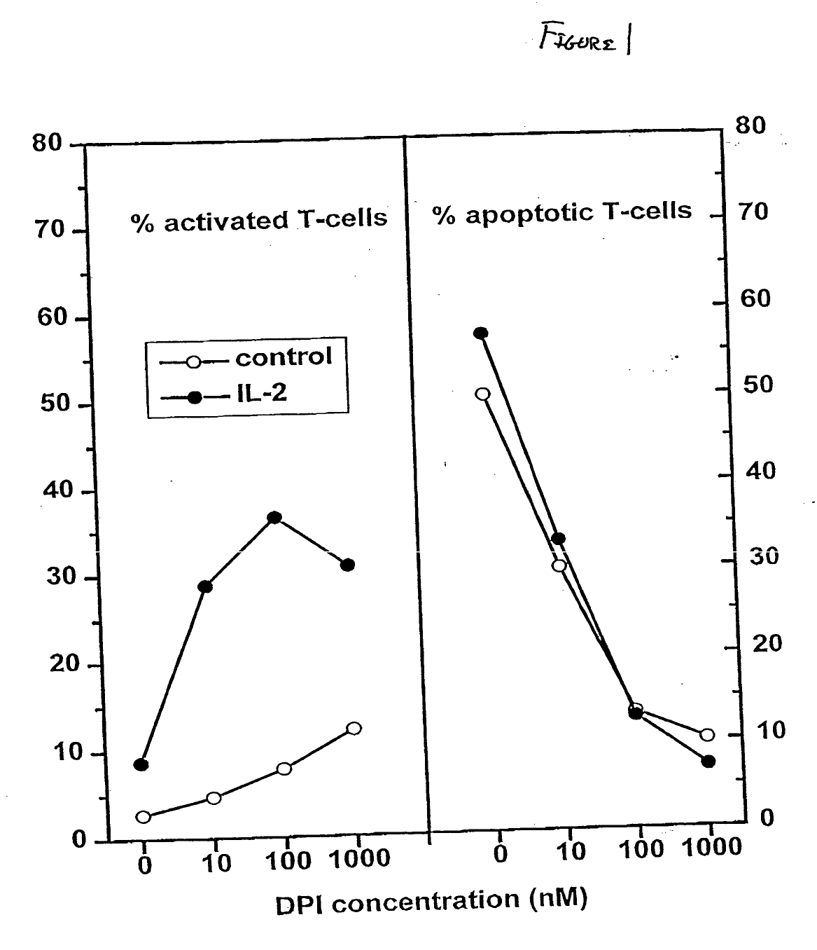

MO can reduce molecular oxygen and generate ROM (respiratory burst), both spontaneously and in response to certain soluble or particulate stimuli (see Klebanoff, S. J., Adv. Host Def. Mech. 1:111-151 (1982)). Experiments were performed in which DPI, an inhibitor of NADPH oxidase activity in MO (Miesel, R., et al., Free. Radic. Biol 20:75-81 (1996)), was added to mixtures of lymphocytes and MO in studies of the acquisition of CD69 on T-cells and NK-cells in response to IL-2. A mixture of MO and lymphocytes was treated with culture medium or IL-2 (100 U/ml) for 16 hours. After incubation, lymphocytes were labeled with antibodies to CD3ε and CD69. Data show CD69 expression in viable T-cells (CD3ε+), and the percentage of T-cells with reduced forward and increased side angle scatter characteristic of apoptosis. Similar results were obtained when CD69 expression was examined in CD56+. NK-cells incubated with MO: 29.5% (control) or 79.0% (DPI 1000 nM) of NK-cells acquired the CD69 antigen in response to IL-2. DPI did not increase the IL-2-induced expression of CD69 in T-cells or NK-cells incubated in the absence of MO. DPI significantly reversed the MO-induced inhibition of T-cells (FIG. 1) and NK-cells (not shown). MO also produce reactive nitrogen intermediates of which nitric oxide (NO) is the ultimate effector molecule, and DPI is an inhibitor also of NO synthetase (Miesel, R., et al., Free. Radic. Biol 20:75-81 (1996)). To study whether NO induction in MO contributed to the observed T- and NK-cell anergy to IL-2, we used a NO synthetase inhibitor, N-monomethyl-L-arginine (L-NMMA). This compound, used at concentrations sufficient to inhibit NO synthesis in MO (Hansson, M, et al., J. Immunol. 156:42-47 (1996)), did not affect the MO-induced suppression of T-cells and NK-cells. Catalase, a scavenger of hydrogen peroxide, significantly reversed the MO-induced inhibition of IL-2-induced CD69 expression in T-cells and NK-cells at concentrations exceeding 50 U/ml, whereas superoxide dismutase, a scavenger of superoxide anion, was ineffective at concentrations sufficient to scavenge >90% of superoxide anion (200 U/ml).

### Example 3

The Fas ligand (CD95L) triggers apoptosis in many cell types after interaction with the Fas receptor (CD95), which is expressed on inter alia T cells (Alderson, M. R., et al., J Exp. Mod. 181:71-77(1995)), and NK-cells (Medvedev, A. E., et al., Cytokine 9:394-404 (1997)). To evaluate the role of FasL/Fas interactions for the observed oxidatively induced apoptosis, a Fas ligand inhibitor was used that comprised the extracellular domain of human Fas (aa 1-154), fused to the Fc portion of human IgG1. This Fas:Fc-IgG fusion protein, used at a concentration (20 μg/ml) sufficient to reduce FasL-mediated, activation-induced apoptosis in T-cells by >60%, did not affect the MO-induced anergy to IL-2 or the MO-induced apoptosis in T-cells or NK-cells (Table 1).

TABLE 1
Fas/FasL-independent anergy and
apoptosis in T-cells and NK-cells.

| | viable | viable | | |
|---|---|---|---|---|
| | CD3+/CD69+ | CD56+/69+ | apoptotic | apoptotic |
| Treatment | (gated | (gated events) | CD3+/ | CD56+/ |
| | events)^{A} | | CD69+(%)^{B} | CD69+(%) |
| control | 89 | 36 | 52 | 71 |
| DPI | 701 | 1248 | 7 | 13 |
| Fas; Fc- | 113 | 26 | 57 | 68 |
| IgG | | | | |

^{A}Lymphocytes and MO were recovered from peripheral blood and labeled with anti-CD3 ε, anti-CD56, and anti-CD69 as described in the legend to table I and analyzed for respective phenotype by flow cytometry. DPI was used at 100 nM and Fas: Fc-IgG at 20 μg/ml.
^{B}Apoptosis was measured by flow cytometry using a gate for lymphocytes with reduced forward scatter and increased right angle scatter. Similar results were obtained in three separate experiments.

### Example 4

The following in vivo study was conducted to determine DPI's effectiveness in inhibiting tumor growth rate. C57/b16 mice were subcutaneously inoculated with mouse sarcoma (MCG) on Day 1. The mice were then subcutaneously injected once daily for Days 1-9 with either NaCl or DPI. There were 5 different treatment groups, including the control group that received NaCl. The other four treatment groups were administered DPI subcutaneously at dosages of either 1 mg/kg, 10 mg/kg, 50 mg/kg, or 100 mg/kg. On Day 9, the mice were killed and tumor size and weight was measured. The resulting tumor weights are provided below in Table 2. The results indicate that DPI has a dose dependent inhibiting effect on tumor growth rate in vivo.

TABLE 2
Mean Tumor Weight (grams) for Mice Treated with DPI

| | | Mean Wt. | | |
|---|---|---|---|---|
| | Count | (gms) | Std. Dev. | Std. Err. |
| DPI 1 mg/kg s.c. | 9 | 1.107 | .497 | .166 |
| DPI 10 mg/kg s.c. | 8 | .643 | .456 | .161 |
| DPI 100 mg/kg s.c. | 9 | .473 | .402 | .134 |
| DPI 50 mg/kg s.c. | 9 | .638 | .326 | .109 |
| Control | 11 | .941 | .355 | .107 |

### Example 5

DPI, in a dose approximately 0.2 to 2.0 mg or 3-10 μg/kg, in a pharmaceutically acceptable form is injected subcutaneously in a sterile carrier solution into subjects in need of enhanced T-cell activity, in this case a patient having a malignancy. Concomitantly, IL-2, for example, human recombinant IL-2 (Proleukin®, Eurocetus), is administered subcutaneously of by continuous infusion of 27 μg/kg/day on days 1-5 and 8-12. This dose represents a total dose of IL-2 considerably lower than that administered by those of skill in the art in connection with cancer therapy.

The above procedure is repeated every 4-6 weeks until an objective regression of tumor disease is observed. The therapy may be continued even after a partial or complete response has been observed. In patients with complete responses, the therapy may be given with longer intervals between cycles.

The treatment may also include periodically boosting patient blood DPI levels by administering 0.2 to 2.0 mg or 3-10 μg/kg of DPI injected 1, 2, or more times per day over a period of one to two weeks at regular intervals, such as daily, bi-weekly, or weekly in order to establish blood DPI at a beneficial concentration

Combination of DPI and Chemotherapeutic Agents

DPI may also be used in conjunction with chemotherapeutic agents to treat a neoplastic or viral disease. Monocyte mediated suppression can be eliminated by administration of DPI prior, during, following or throughout chemotherapy in order to facilitate activation and protection of cytotoxic lymphocytes.

Representative compounds used in cancer and antiviral therapies are described above. Other cancer and antiviral therapeutic compounds can also be utilized. Similarly, malignancies and viral diseases against which the treatment herein may be effective, and thus may be directed, are also described. It should be noted that the amounts, routes of administration and dosage protocols for these cancer and antiviral compounds used are well known to those of skill in the art. The teaching herein are also directed toward augmenting the efficacy of these compounds, and the therapeutic results of their use. Therefore, the conventional methodologies for their use, in conjunction with the compounds and methods provided herein, are contemplated as sufficient to achieve a desired therapeutic effect.

### Example 6

Subjects in need of enhanced cytotoxic lymphocyte activity, because of a neoplastic disease, and/or a viral infection such as hepatitis B (HBV), hepatitis C(HCV), human immunodeficiency virus (HIV), human papilloma virus (HPV) or herpes simplex virus (HSV) type 1 or 2, or other viral infections, are administered human recombinant IL-2 (Proleukin®, Eurocetus) by subcutaneous injection or by continuous infusion of 27 μg/kg/day on days 1-5 and 8-12. Additionally, subjects also receive a daily dose of 6×10⁶ U interferon-α administered by a suitable route, such as subcutaneous injection. This treatment also includes administering 0.2 to 2.0 mg or 3-10 μg/kg of DPI injected 1, 2, or more times per day in conjunction with the administration of IL-2 and/or interferon-α.

The above procedure is repeated every 4-6 weeks until an objective regression of the tumor is observed, or until improvement in the viral infection occurs. The therapy may be continued even after a first, second, or subsequent complete remission has been observed. In patients with complete responses, the therapy may be given with longer intervals between cycles.

The treatment may also include periodically boosting patient blood DPI levels by administering 0.2 to 2.0 mg or 3-10 μg/kg of DPI injected 1, 2, or more times per day over a period of one to two weeks at regular intervals, such as daily, bi-weekly, or weekly in order to establish or maintain blood DPI at a beneficial concentration, e.g., at a concentration above 0.21 mole/L.

Additionally, the frequency of interferon-α administration may be varied depending on the patient's tolerance of the treatment and the success of the treatment. For example, interferon may be administered three times per week, or even daily, for a period of up to 24 months. Those skilled in the art are familiar varying interferon treatments to achieve both beneficial results and patient comfort.

### Example 7

Subjects with AML in a first, second, subsequent or complete remission are treated in 21-day courses with IL-2 [35-50 μg (equivalent to 6.3-9×10⁵ IU) subcutaneously (s.c.). twice daily], repeated with three to six-week intermissions and continued until relapse. In cycle #1, patients receive three weeks of low dose chemotherapy consisting of 16 mg/m²/day cytarabine, and 40 mg/day thioguanine. Concomitantly, patients are injected subcutaneously with 0.2 to 2.0 mg or 3-10 μg/kg of a pharmaceutically acceptable form of DPI to boost circulating DPI to a beneficial level twice daily (above 0.2 μmole/L). DPI levels may be continually boosted to beneficial levels by administering DPI by injection at 0.2 to 2.0 mg or 3-10 μg/kg twice daily in a pharmaceutically acceptable form of a ROM inhibitory compound during the IL-2 treatment. Thereafter, the subjects are allowed to rest for three to six weeks.

After the rest period at the end of the first cycle (cycle #1), the second cycle (cycle #2) is initiated. Twice daily, injections of a pharmaceutically acceptable form of a ROM inhibitory compound in a sterile carrier solution are administered at 0.5 to 2.0 mg or 3-10 μg/kg subcutaneously. Cytarabine (16 mg/m²/day s.c.) and thioguanine (40 mg/day orally) are given for 21 days (or until the platelet count is <50×10^{9/1}). In the middle week, patients receive 0.2 to 2.0 mg or 3-10 μg/kg per injection twice per day of a pharmaceutically acceptable form of DPI to boost circulating DPI to beneficial levels. At the end of the three week chemotherapy treatment, patients receive 0.2 to 2.0 mg or 3-10 μg/kg per injection twice daily of a pharmaceutically acceptable form of DPI for a week. Thereafter, patients receive interleukin-2 for three weeks. Patients are permitted to rest for three to six weeks.

Thereafter, cycle #3 is initiated. Cycle #3 is identical to cycle #2.

Alternatively, the treatment may also include periodically boosting patient blood DPI levels by administering 0.2 to 2.0 mg or 3-10 μg/kg of DPI injected 1, 2, or more times per day over a period of one to two weeks at regular intervals, such as daily, bi-weekly, or weekly in order to achieve a beneficial blood DPI concentration. Another alternative is to provide DPI in a depot or controlled release form.

### DISCUSSION

The data presented herein demonstrate that MO inhibit cytotoxic lymphocyte activation. MO inhibition of cytotoxic lymphocyte activation appears to be mediated by ROM formation. The experiments discussed above show that MO inhibition of cytotoxic lymphocyte is reversed through the addition of a ROM inhibitory compound such as DPI. These results suggest that cytotoxic lymphocyte activation may benefit from a down-regulation of MO inhibition.

### CONCLUSION

While particular embodiments of the teaching herein have been described in detail, it will be apparent to those of skill in the art that these embodiments are exemplary, rather than limiting. All references are hereby expressly incorporated by reference.

What is claimed is:

1. A method of protecting cytotoxic T lymphocytes and NK cells in a subject, for the treatment of tumors, viral diseases or inflammatory diseases, comprising:
identifying a subject in need of cytotoxic T lymphocyte and NK cell enhancement; and
administering to the subject an amount of NADPH-oxidase inhibitor effective to protect cytotoxic T lymphocytes and NK cells in the presence of monocytes or macrophages.

2. The method of claim 1, wherein said NADPH-oxidase inhibitor is diphenyliodonium (DPI).

3. The method of claim 2, wherein said amount of DPI is selected from a daily dose between about 10 to 100 mg/kg.

4. The method of claim 3, wherein said amount of DPI is administered at a daily dose of about 10 mg/kg.

5. The method of claim 3, wherein said amount of DPI is administered at a daily dose of about 50 mg/kg.

6. The method of claim 3, wherein said amount of DPI is administered at a daily dose of about 100 mg/kg.

7. The method of claim 1, further comprising administering an effective amount of a cytotoxic lymphocyte stimulatory composition to the subject, wherein said cytotoxic lymphocyte stimulatory composition is selected from the group consisting of a vaccine adjuvant, a vaccine, a peptide, a cytokine, and a flavonoid.

8. The method of claim 7, wherein the composition is a cytokine selected from the group consisting of IL-1, IL-2, IL-12, IL-15, IFN-α, IFN-β, and IFN-γ.

9. The method of claim 7, wherein the composition is a flavonoid selected from the group consisting of flavone acetic acids and xanthenone-4-acetic acids.

10. The method of claim 7, wherein said cytotoxic lymphocyte stimulatory composition is administered in a daily dose of between 1,000 and 600,000 U/kg.

11. The method of claim 1, further comprising administering of an effective amount of a compound that inhibits the production or release of intercellular reactive oxygen metabolites (ROM) selected from the group consisting of histamine, histamine dihydrochloride, histamine phosphate, serotonin, dimaprit, clonidine, tolazoline, impromadine, 4-methylhistamine, betazole, and a histamine congener.

12. The method of claim 11, wherein said effective amount of a compound that inhibits the production or release of intercellular reactive oxygen metabolites is between 0.05 and 50 mg per dose.

13. The method of claim 11, wherein said effective amount of a compound that inhibits the production or release of intercellular reactive oxygen metabolites is between 1 and 500 μg/kg of patient weight per dose.

14. The method of claim 11, wherein the administration of said NADPH-oxidase inhibitor and said compound that inhibits the production or release of intercellular reactive oxygen metabolites (ROM) is performed within 1 hour.

15. The method of claim 11, wherein the administration of said NADPH-oxidase inhibitor and said compound that inhibits the production or release of intercellular reactive oxygen metabolites (ROM) is performed within 24 hours.

16. The method of claim 11, wherein said intercellular reactive oxygen metabolite is hydrogen peroxide.

17. The method of claim 11, further comprising administering an effective amount of a ROM scavenger.

18. The method of claim 17, wherein said ROM scavenger is selected from the group consisting of catalase, glutathione peroxidase, vitamin E, vitamin A, vitamin C, SOD, SOD mimetics, and ascorbate peroxidase.

19. The method of claim 17, wherein said ROM scavenger is administered in a dose of from about 0.05 to about 50 mg/day.

20. The method of claim 17, wherein said effective amount of NADPH-oxidase inhibitor, said compound that inhibits the production or release of intercellular reactive oxygen metabolites, and said ROM scavenger are administered separately.

21. The method of claim 1, further comprising administering a chemotherapeutic agent.

22. The method of claim 21, wherein the chemotherapeutic agent comprises an anticancer agent selected from the group consisting of cyclophosphamide, chlorambucil, melphalan, estramustine, iphosphamide, prednimustin, busulphan, tiottepa, carmustin, lomustine, methotrexate, azathioprine, mercaptopurine, thioguanine, cytarabine, fluorouracil, vinblastine, vincristine, vindesine, etoposide, teniposide, dactinomucin, doxorubin, dunorubicine, epirubicine, bleomycin, nitomycin, cisplatin, carboplatin, procarbazine, amacrine, mitoxantron, tamoxifen, nilutamid, and aminoglutemide.

23. The method of claim 21, wherein administering said effective amount of NADPH-oxidase inhibitor and said chemotherapeutic agent are performed concomitantly.

24. A composition to protect cytotoxic T lymphocytes and NK cells in a subject, for the treatment of tumors, viral diseases or inflammatory diseases, comprising an effective amount of NADPH-oxidase inhibitor in a pharmaceutically acceptable carrier.

25. The composition of claim 24, wherein said NADPH-oxidase inhibitor is diphenyliodonium (DPI).

26. The composition of claim 24, further comprising a cytotoxic lymphocyte stimulatory compound selected from the group consisting of a vaccine adjuvant, a vaccine, a peptide, a cytokine, and a flavonoid.

27. The composition of claim 26, wherein the compound is a cytokine selected from the group consisting of IL-1, IL-2, IL-12, IL-15, IFN-α, IFN-β, and IFN-γ.

28. The composition of claim 26, wherein the compound is a flavonoid selected from the group consisting of flavone acetic acids and xanthenone-4-acetic acids.

29. The composition of claim 26, wherein said cytotoxic lymphocyte stimulatory composition is administered in a daily dose of between 1,000 and 600,000 U/kg.

30. The composition of claim 24, further comprising an effective amount of a compound that inhibits the production or release of intercellular reactive oxygen metabolites (ROM) selected from the group consisting of histamine, histamine dihydrochloride, histamine phosphate, serotonin, dimaprit, clonidine, tolazoline, impromadine, 4-methylhistamine, betazole, and a histamine congener.

31. The composition of claim 30, wherein said effective amount of a compound that inhibits the production or release of intercellular reactive oxygen metabolites (ROM) is between 0.05 and 50 mg per dose.

32. The composition of claim 30, wherein said effective amount of a compound that inhibits the production or release of intercellular reactive oxygen metabolites (ROM) is between 1 and 500 μg/kg of patient weight per dose.

33. The composition of claim 24, further comprising a chemotherapeutic agent.

34. The composition of claim 33, wherein the chemotherapeutic agent comprises an anticancer agent selected from the group consisting of cyclophosphamide, chlorambucil, melphalan, estramustine, iphosphamide, prednimustin, busulphan, tiottepa, carmustin, lomustine, methotrexate, azathioprine, mercaptopurine, thioguanine, cytarabine, fluorouracil, vinblastine, vincristine, vindesine, etoposide, teniposide, dactinomucin, doxorubin, dunorubicine, epirubicine, bleomycin, nitomycin, cisplatin, carboplatin, procarbazine, amacrine, mitoxantron, tamoxifen, nilutamid, and aminoglutemide.

35. The composition of claim 25, wherein said effective amount DPI is selected from a daily dose between about 10 to 100 mg/kg.

36. The composition of claim 25, wherein said effective amount of DPI is administered at a daily dose of about 10 mg/kg.

37. The composition of claim 25, wherein said effective amount of DPI is administered at a daily dose of about 50 mg/kg.

38. The composition of claim 25, wherein said effective amount of DPI is administered at a daily dose of about 100 mg/kg.
